(19) United States
(12) Patent Application Publication
(10) Pub. No.: US 2014/0188108 A1
(43) Pub. Date: Jul. 3, 2014

(54) Energy Assisted Tissue Piercing Device and Method of Use Thereof

(71) Applicant: Mitralign, Inc., Tewksbury, MA (US)

(72) Inventors: Dennis Goodine, Dracut, MA (US); George Purtell, Westford, MA (US); Steven D. Cahalane, Pelham, NH (US); Paul T. Modoono, Chelmsford, MA (US); John P. Brunelli, North Attleboro, MA (US); Edward I. McNamara, Chelmsford, MA (US)

(73) Assignee: Mitralign, Inc., Tewksbury, MA (US)

(21) Appl. No.: 14/138,926

(22) Filed: Dec. 23, 2013

Related U.S. Application Data
(60) Provisional application No. 61/747,196, filed on Dec. 28, 2012.

(51) Int. Cl. A61B 18/14

(52) U.S. Cl. CPC A61B 18/1492
USPC 606/45

(57) ABSTRACT

An energy assisted tissue piercing device that enables a tissue piercing wire to puncture heart tissue percutaneouly. The energy assisted tissue piercing device includes an outer delivery catheter, an inner tissue piercing wire, and an energy source. The inner tissue piercing wire is disposed longitudinally through the lumen of the outer delivery catheter during delivery. The inner needle has a flexible portion that allows the wire to bend when it is free from the constraint of the outer delivery catheter. The inner tissue piercing wire is conductive and connected to an energy source and the piercing of the heart tissue is done with the aid of the energy from the energy source.

### CROSS REFERENCE TO RELATED APPLICATION

The present application claims the benefit of U.S. patent application Ser. No. 61/747,196, filed Dec. 28, 2012, which is hereby incorporated by reference in its entirety.

### TECHNICAL FIELD

The present teachings generally relate to an energy assisted tissue piercing device, for example, for piecing heart valve tissue. The present teachings also relate to using a delivery system with such an energy assisted tissue penetration device to create an aperture percutaneously.

### BACKGROUND

Transeptal puncture is a commonly performed procedure that allows access to the left atrium. The most common uses for transeptal catheterization include direct measurement of the left atrial pressure or providing access to the left ventricle in patients with prosthetic aortic or mitral valves or in patients who are undergoing percutaneous mitral valvuloplasty etc.

Historically, conventional rigid, long needles, such as Brockenbrough or Ross needles, are used for this procedure to mechanically puncture the atrial septum. And a challenge for a successful transept puncture with a Brockenbrough needle is to position the Brockenbrough needle at the thinnest aspect of the atrial septum, the membranous fossa ovalis. Although the procedure is generally safe, serious complications, such as inadvertent puncture through tissues other than the septum, for example, the atrial free wall, pose a significant risk to the patient.

Many companies are working to improve the drawback associated with the Brockenbrough needle. St. Jude Medical Inc.'s ACross™ Transeptal Access System, which consolidates a sheath, a dilator and a needle into a single interlocking handle, gives a clinician a greater control over precisely positioning the device and ensures that the puncture needle is only advanced for a predetermined distance. Baylis Medical's NRG™ RF Transeptal Needle uses radiofrequency energy emitted from the needle tip to assist the needle in transeptal access. NRG™ RF needle is insulated with a closed end that safely delivers radiofrequency energy to create a small hole in the atrial septum, allowing the needle to pass to the left atrium with increased efficacy and control. Pressure Product's SafeSept™ septal puncture system includes a delivery catheter and a sharp tipped wire. When supported by the delivery catheter, the sharp tip of the wire ensures an effortless penetration of the septal tissue. When unsupported by the delivery catheter, the tip of the wire assumes a ‘J’ shape, rendering it incapable of penetrating tissues.

The left atrium is the most difficult chamber to access percutaneously. In addition, most septal puncture location is applied at the fossa ovalis, the thinnest part of the atrial septum. Thus there exists a need for a clinician to safely penetrate heart tissue percutaneously at any treatment location, whether it is the thickest or the thinnest, or intact or scarred tissue. In addition, an ideal transeptal puncture system should be able to precisely control the puncture location, avoid needle slippage and inadvertent puncturing, and is easy for a clinician to practice.

### SUMMARY

One aspect of the present teachings provides a device for piercing heart tissue percutaneously. The device comprises a first outer catheter, a second inner tissue piercing wire, and an energy source connecting to the second inner tissue piercing wire. The first outer catheter has a proximal end, a distal end and a lumen longitudinally disposed therethrough. The second inner tissue piercing wire has a conductive inner wire and an insulation outer layer. The second inner tissue piercing wire slidably disposes within the lumen of the outer catheter. The conductive inner wire of the second inner tissue piercing wire has a proximal portion, a distal portion, and an intermediate portion. The intermediate portion is narrower than the proximal portion and distal portion of the conductive inner wire.

Another aspect of the present teachings also provides a device for piercing heart tissue percutaneously. The device comprises a first outer catheter, a second inner tissue piercing wire, and an energy source connecting to the second inner tissue piercing wire. The first outer catheter has a proximal end, a distal end and a lumen longitudinally disposed therethrough. The second inner tissue piercing wire has a proximal portion, a distal portion and an intermediate portion between the proximal and distal portions. The second inner tissue piercing wire is configured to transition from a delivery profile to a deployed profile. In the delivery profile, the distal portion of the second inner tissue piercing wire is disposed within the lumen of the first outer catheter, and substantially aligns with the proximal portion of the second inner tissue piercing wire. In the deployed profile, the distal portion is exposed outside of the lumen of the first outer catheter, and pivots from the proximal portion of second inner tissue piercing wire.

### BRIEF DESCRIPTION OF THE DRAWINGS

FIG. 1 is a perspective view of an exemplary energy assisted tissue piecing device in accordance with the present teachings;

FIG. 2 is a perspective view of an exemplary energy assisted tissue piecing device in accordance with the present teachings;

FIG. 3a is a perspective view of an exemplary tissue piercing wire in accordance with the present teachings;

FIG. 3b is a perspective view of an exemplary tissue piercing wire in accordance with the present teachings;

FIG. 4 is a perspective view of an exemplary conductive inner wire in accordance with the present teachings;

FIG. 5 is a perspective view of an exemplary conductive inner wire in accordance with the present teachings;

FIG. 6 is a perspective view of an exemplary conductive inner wire in accordance with the present teachings;

FIG. 7 is a perspective view of an exemplary energy assisted tissue piecing device in accordance with the present teachings;

FIG. 8a is a perspective view of an exemplary energy assisted tissue piecing device deploying across a tissue in accordance with the present teachings; and

FIG. 8b is a perspective view of an exemplary energy assisted tissue piecing device deploying across a tissue in accordance with the present teachings.

### DETAILED DESCRIPTION OF CERTAIN EMBODIMENTS

Certain specific details are set forth in the following description and Figures to provide an understanding of various embodiments of the present teachings. Those of ordinary skill in the relevant art will understand that they can practice other embodiments of the present teachings without one or more of the details described below. Thus, it is not the intention of the Applicants to restrict or in any way limit the scope of the appended claims to such details. While various processes are described with reference to steps and sequences in the following disclosure, the steps and sequences of steps should not be taken as required to practice all embodiments of the present teachings.

As used herein, the term “lumen” means a canal, a duct, or a generally tubular space or cavity in the body of a subject, including a vein, an artery, a blood vessel, a capillary, an intestine, and the like.

As used herein, the term “proximal” shall mean closest to the operator (less into the body) and “distal” shall mean furthest from the operator (further into the body). In positioning a medical device inside a patient, “distal” refers to the direction away from a catheter insertion location and “proximal” refers to the direction nearer the insertion location.

As used herein, the term “wire” can be a strand, a cord, a fiber, a yarn, a filament, a cable, a thread, or the like, and these terms may be used interchangeably.

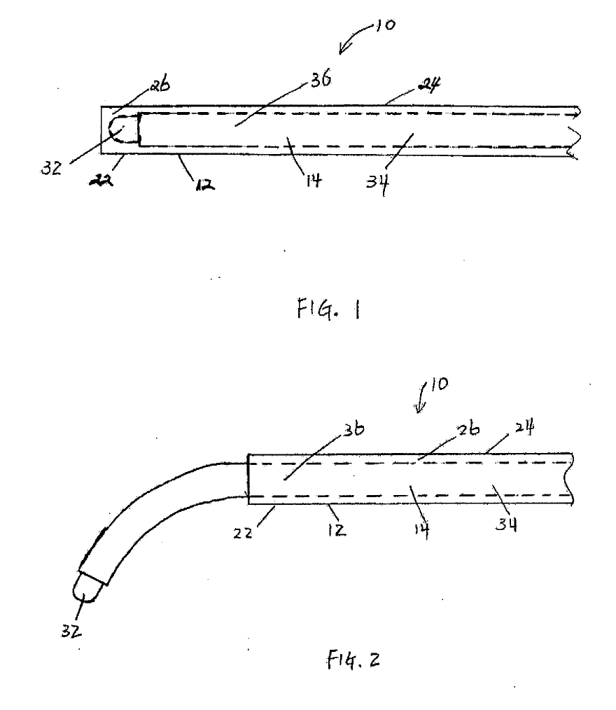

The following description refers to FIGS. 1 to 8. A person with ordinary skill in the art would understand that the figures and description thereto refer to various embodiments of the present teachings and, unless indicated otherwise by their contexts, do not limit the scope of the attached claims.

Unless otherwise specified, all numbers expressing quantities, measurements, and other properties or parameters used in the specification and claims are to be understood as being modified in all instances by the term “about.” Accordingly, unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The present teachings described herein relates to devices and methods for puncturing the mitral annulus via a percutaneous route. The devices and/or methods can be used to repair the mitral valve, for example, to treat the mitral regurgitation or other related mitral valve diseases. It, however, should be appreciated that the present teachings are also applicable for other parts of the anatomy or for other indications. For instance, a device such as one described in the present teachings can be used to puncture the atrial septum, the tricuspid annulus and etc.

One aspect of the present teachings relates to an energy assisted tissue piercing device and methods of using such a device to percutaneously penetrate the mitral armulus. In some embodiments, the device creates a controlled perforation with the distal tip through the application of radio frequency (RF) energy. In some embodiments, the device has a generally straight profile before perforation. In some embodiments, the device has a curved profile upon entering the other side of the annulus tissue. The curved profile can direct the distal tip of the device away from the cardiac structures, thereby decreasing the likelihood of injury, including inadvertent cardiac perforation.

According to some embodiments of the present teachings, the energy assisted tissue piecing device includes a delivery catheter, a tissue piercing wire, and an energy source connected to the tissue piercing wire. The delivery catheter has a proximal portion, a distal portion, and an elongated lumen extending from the proximal portion to the distal portion. The tissue piercing wire also has a proximal end, a distal end, and an elongated body in between. The tissue piercing wire includes an insulated body and an un-insulated distal tip. In some embodiments, the tissue piercing wire has a deflectable distal portion. In some embodiments, the ability of the piercing device to deflect is achieved by using a flexible material or a shape memory material connecting the distal portion to the rest of the wire. Alternatively, the ability to deflect is achieved with geometrical modifications of the wire.

FIG. 1 is a plane view of the distal portion of an energy assisted tissue piecing device (10) according to an embodiment of the present teachings. The exemplary percutaneous device (10) includes a delivery catheter (12), a tissue piercing wire (14), and an energy source (not shown) connected to the proximal portion (not shown) tissue piercing wire (14). The delivery catheter (12) in the exemplary percutaneous device has a proximal end (not shown), a distal end (22), an elongated body (24) between the proximal and distal ends, and a lumen (26) axially disposed along the long axis of the delivery catheter (12). In certain embodiments, the delivery catheter (12) has a general diameter of 2-3 mm.

The tissue piercing wire (14) of some embodiments is axially disposed within the lumen (26) of the delivery catheter (12) as illustrated in FIG. 1. The tissue piercing wire (14) also includes a proximal end (not shown), a distal end (32), and an elongated body (34). The tissue piercing wire (14) is reciprocally and axially moveable in the lumen (26) of the delivery catheter (12). And the tissue piercing wire (14) can be rotated as well. The device has a delivery profile where a distal end portion (36) of the tissue piercing wire (14) resides inside the lumen (26) of the delivery catheter (12) as illustrated in FIG. 1. According to some embodiments of the present teachings, when the distal end portion (36) of the tissue piercing wire (14) is enclosed within the lumen (26) of the delivery catheter (12), the entire length of the tissue piercing wire (14) is substantially straight and parallels the longitudinal axis of the delivery catheter (12). The device also has a deployed profiled where the distal end portion (36) of the tissue piercing wire extends distally outside the lumen (26) of the delivery catheter (12) as illustrated in FIG. 2. According to some embodiments of the present teachings, when the distal end portion (36) of the tissue piercing wire (14) is free from the constrain of the delivery catheter (12), the distal end portion (36) of the tissue piercing wire (14) bends radially away from the longitudinal axis of the wire.

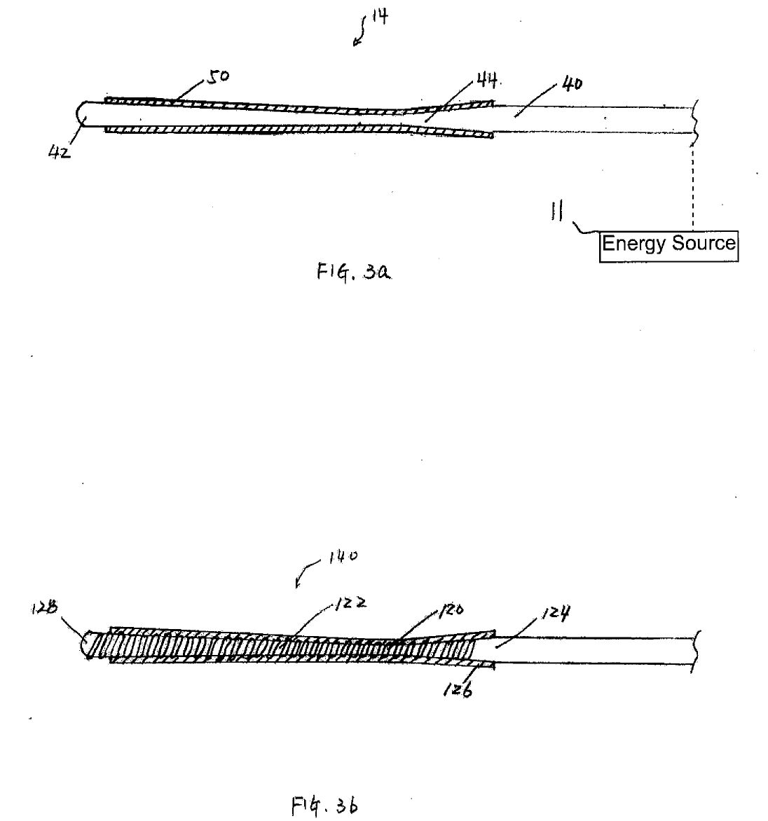

FIG. 3a illustrates a detailed construction of an exemplary tissue piercing wire (14) according to one embodiment of the present teachings. The tissue piercing wire (14) includes a conductive inner wire (40), and an insulation outer layer (50). FIG. 3a illustrates a specific embodiment where the insulation outer layer (50) covers a portion (44) of the conductive inner wire (40) proximal to the distal end (42) of the conductive inner wire (40) leaving the distal end (42) of the conductive inner wire (40) uninsulated. During deployment, the insulation outer layer (50) insulates the conductive inner wire (40) from heating up the blood when this portion of the conductive inner wire (40) slides outside of the delivery catheter (12) during deployment. One skilled in the art should understand that the insulation outer layer (50) can covers the entire length of the conductive inner wire (40) from its proximal end and leaving only its distal end (42) exposed. In one embodiment, the insulation outer layer (50) is made of material such as PTFE. In another embodiment, the insulation layer is in the form of a coating, a temporary sleeve, a permanent sleeve, or an extrusion over the inner wire (40). Optionally, other conventional methods can also be used to form the nonconductive layer.

According to another embodiment of the present teachings as illustrated in FIG. 3b, a tissue piercing wire (140) could also include a coil spring (120) covering a portion (122) of the conductive inner wire (124) from its distal end (128). In one embodiment, the coil spring (120) lays between the conductive inner wire (124) and the insulation outer layer (126). One skilled in the art should understand that the coil spring (120) could cover the entire length of the conductive inner wire (124). Additionally, the coil spring (120) could be attached to the conductive inner wire (124) with many means known in the field, such as welding, gluing, soldering, and etc.

Still referring to FIG. 3a, the inner wire (40) has an un-insulated distal tip (42). In some embodiments of the present teachings, the un-insulated distal tip is in the shape of a half-sphere. One ordinarily skilled in the art would understand that the distal tip (42) of the inner wire (40) can have other shapes and configurations.

In some embodiments of the present teachings, the conductive inner wire is made of a material conventionally used for guide wires. Examples of the material include a straight stainless steel wire, a coiled stainless steel wire, a glass fiber, a plastics material, nitinol, and etc. In other embodiments, the insulation layer is made of a non-conductive polymer, such as a polyimide, PEBAX®, a polyethylene, a polytetrafluoroethylene (PTFE), a poly(fluorinatedethylenepropylene) (FEP), and a polyurethane, and etc.

According to some embodiments, the proximal end of the tissue piercing wire (14) is connected to an energy source (11). The energy source (11) can provide one or more energy types, including, but not limited to, microwave, infrared, visible light, ultraviolet rays, x-rays, gamma rays, cosmic rays, acoustic energy, thermal energy, or radio frequency energy. In certain embodiments, the energy source (11) is radio frequency energy (RF). For example, the energy source (11) is connected directly to the tissue piercing wire (14) as illustrated in FIG. 3(a). In certain other embodiments, the energy source (11) is connected to the delivery catheter (12) or a component of the delivery catheter (12) to which the tissue piercing wire (14) is connected. Alternative modes of coupling the energy source (11) to the tissue piercing wire (14) will be obvious to one of ordinary skill in the art and are within the scope of the invention.

According to some embodiments, the delivery catheter (12) provides structural support for the tissue piercing wire (14). In some embodiments, the delivery catheter (12) also functions as a dilator. In some embodiments, the inner diameter of the lumen of the delivery catheter (12) typically approximates the outer diameter of the tissue piercing wire (14) such that the delivery catheter (12) provide support to the wire during crossing.

In some embodiments, the device may further comprise a delivery sheath through which the device passes from outside the patient's body through a vessel. In some embodiments, the device comprises a control handle at the proximal end of the sheath. The sheath and/or other components of the delivery system may be steerable by using actuators (not shown) on the control handle to aid in delivering and deploying the device along the tortuous vascular path leading to the treatment site.

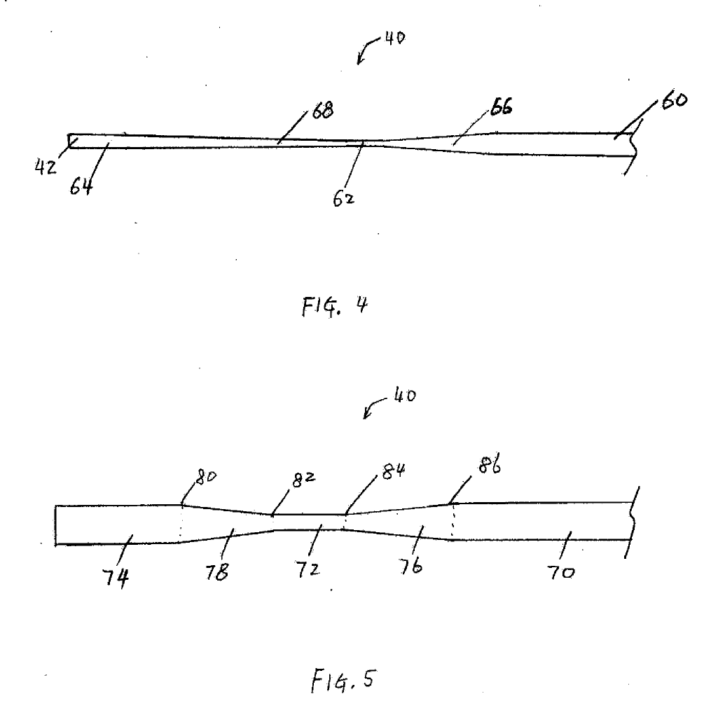

FIG. 4 is a longitudinal view of the conductive inner wire (40) of the tissue piercing wire (14) of an exemplary transeptal puncture device according to the present teachings. The exemplary inner wire (40) includes a proximal portion (60), an intermediate portion (62), and a distal portion (64). The intermediate portion of the inner wire (40) is narrower in diameter than the proximal (60) and distal portions (64) of the inner wire (40). In some embodiments, the inner wire (40) has graduate and/or tapered transitions (66, 68) between the intermediate (62) and distal (64) or proximal portions (60) as illustrated in FIG. 4. Alternatively, the change in outer diameter from the distal or proximal portions (60, 64) to the intermediate portion (62) is abrupt.

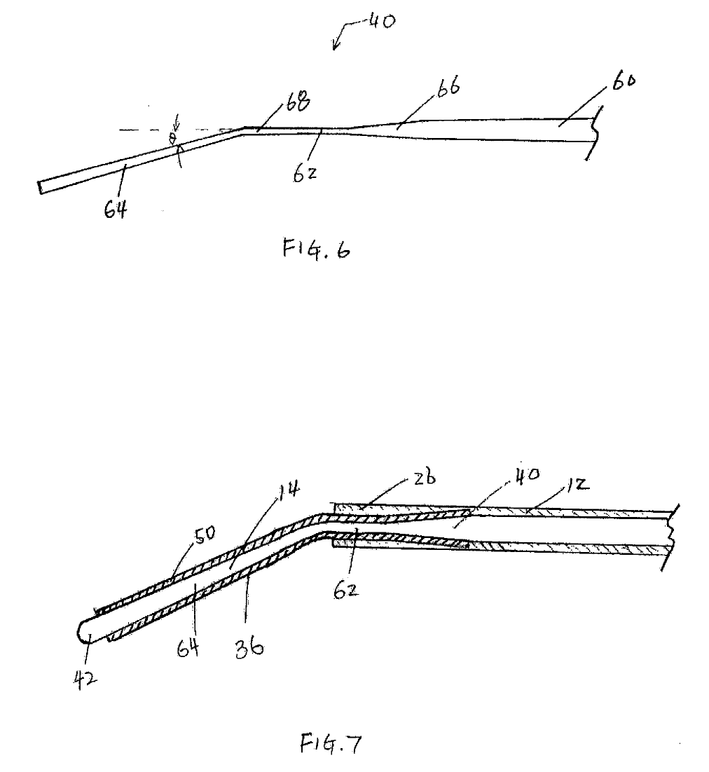

According to some embodiments, the narrower intermediate portion (62) of the inner wire (40) allows the inner wire (40) to have a greater flexibility or bendability than the remaining portions of the wire. This narrower intermediate portion (62) can allow the distal portion (64) of the wire to bend when the distal portion (64) of the inner wire (40) extends outside the delivery catheter (12) (as described when referring to FIG. 7) and prevent the distal end (42) of the conductive inner wire (40) from causing damage to the unintended places inside a heart.

According to some embodiments, the length of the distal portion (64) is from about 5 mm to about 30 mm. In some embodiments, the length of the intermediate portion (62) is from about 5-15 mm. Preferably, the distal portion (64) is 11-13 mm long and the intermediate portion (62) is 12-14 mm long. In other embodiments, the transitioning portions (66, 68) between the intermediate and the proximal (60) or distal (64) portion is 3-63 mm long.

According to some embodiments of the present teachings, the distal and proximal portions (60, 64) of the inner wire (40) have the same diameter as illustrated in FIG. 4. In one embodiment, the inner wire (40) has an outer diameter of 0.2 mm to 1 mm, and the intermediate portion (62) has a general diameter of 0.1-0.5 mm. Alternatively, the intermediate portion (62) has a general diameter 20-80% smaller than the general diameter of the inner wire (40).

Alternatively, the distal and proximal portions (60, 64) of the inner wire (40) have different diameters. In some embodiments, the distal portion (64) is narrower than the proximal portion (60). In other embodiments, the proximal portion (60) is narrower than the distal portion (64). In certain embodiments, one of the proximal and distal portions (60, 64) has an outer diameter 40-60% smaller than the general diameter of the other portions.

FIG. 5 illustrates an exemplary embodiment of the inner wire (40) according to the present teachings. In this embodiment, the inner wire (40) has a distal portion (74), a distal tapered transition portion (78), an intermediate portion (72), a proximal tapered transition portion (76), and a proximal portion (70). The distal portion (74) of the inner wire (40) can have a diameter of about 0.1-0.3 mm and a length of 11-13 mm. The diameter of the intermediate portion (72) can be 5-70% of the diameter of the distal portion (74). The length of the intermediate portion (74) can range from 11-13 mm. The proximal portion (70) of the inner wire (40) has a diameter of 0.25-0.45 mm. One skilled in the art should understand that the length of the proximal portion (70) is determined by the needs of the application.

Continues referring to FIG. 5, the distal tapered transition (78) portion has a length of 61-63 mm. The distal end (80) of the distal tapered transition portion (78) joins the distal portion (74) and has the same diameter of the distal portion (74) of the inner wire (40). The proximal end (82) of the distal tapered transition portion (78) joins the intermediate portion (72) and has the same diameter of the intermediate portion (72) of the inner wire (40). The proximal tapered transition portion (76) has a length of 2.8-3.1 mm. The proximal end (86) of the proximal tapered transition portion (76) joins the proximal portion (70) and has the same diameter of the proximal portion (70) of the inner wire (40). The distal end (84) of the proximal tapered transition portion (76) joins the intermediate portion (72) and has the same diameter of the intermediate portion (72) of the inner wire (40). According to some embodiments of the present teachings, the diameter of the distal and proximal tapered transition portions (76, 78) reduces gradually from one end to the other.

In some embodiments of the present teachings, the intermediate portion (72), and/or proximal portion (70) of the inner wire (40) is made by removing material from a typical guide wire known by those with ordinary skill in the field. Methods of removing material from the guide wire include grinding, milling, and etc.

In some embodiments, the distal portion (74) of the inner wire (40) may be straight (e.g., 0 degrees) as illustrated in FIG. 4 or bent at an angle ranging from about >0 degree to about 270 degree as illustrated in FIG. 6.

Now referring to FIG. 7, according to some embodiments, when the distal portion of the tissue piercing wire (14) is not constrained within the lumen (26) of the delivery catheter (12), the distal end portion (36) of the tissue piercing wire (14) turns radially away from the longitudinal axis of the wire (14) and provides an essentially non-traumatic conformation. In certain embodiments, the bending point(s) is at the intermediate portion (62) of the conductive inner wire (40) as illustrated in FIG. 7. In certain other embodiments, the wire (14) can bend at the transition portion next to the intermediate portion (62).

According to some embodiments, the device penetrates heart tissue with the use of radio frequency energy. Preferably, unipolar electrodes can be used for the inner conductive wire with grounding pads typically placed on the patient's thighs. Alternatively, a bipolar electrode system can be employed as well. The application of radio frequency energy to the inner wire (40) increases the tissue temperature around the distal tip of the inner wire (40) to over 100° C. Mechanical cohesion in the tissue is then diminished, allowing the distal portion of the inner wire (40) to advance as pressure is applied to the tissue by a clinician from the proximal end of the device. In an alternative embodiment, any other methods producing heat (e.g., such as electrical resistance, laser, or ultrasound) can also be used. In some embodiments, the incision is created slowly to reduce the risk of accidental puncture of tissue elsewhere.

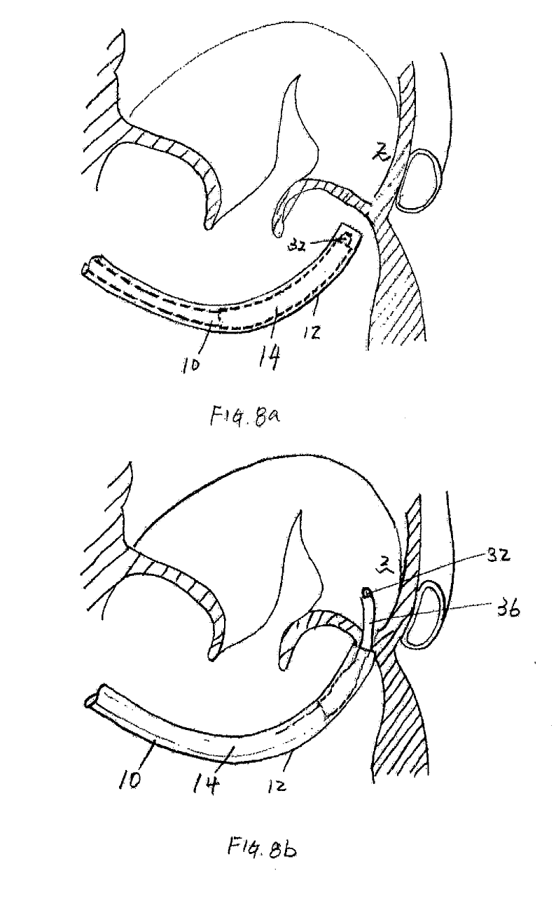

Now referring to FIGS. 8a-b, a tissue piercing device (10) is delivered and deployed to a mitral annulus (2) according to one embodiment of the present teachings. What is described below are certain exemplary percutaneous delivery methods of the tissue piercing device (10) of the present teachings. One ordinarily skilled in the art would understand that other ways of percutaneous delivery can also be used without departing from the spirit of the present teachings. Thus the disclosure below should not be viewed limiting. The tissue piercing device (10) described herein can also be used to pierce holes at other location in the heart. For example, the tissue piercing device (10) described herein can be used to create incision through the atrial septal wall, the ventricle wall, chronic total occlusion, mitral annulus, tricuspid annulus, and etc.

According to one embodiment of the present teachings, a delivery sheath is directed into the aorta, through the aortic valve and into the left ventricle between the cordae tendonae. This delivery sheath is then used as a conduit for the tissue piercing device (10) to be delivered to the treatment site. One skilled in the art should understand, a delivery sheath may not be necessary, and thus the tissue piercing device (10) can be directly advanced to the treatment location.

FIG. 8a illustrates a tissue piercing device (10) being delivered to the mitral annulus (2). According to certain embodiments of the present teachings, a tissue piercing device (10) is in its delivery profile where the distal end (32) of the tissue piercing wire (14) is constrained within the delivery catheter (12) and the distal end (32) of the tissue piercing wire (14) is inside the lumen of the delivery catheter (12). In one embodiment of the present teachings, the tissue piercing device (10) advances through the longitudinal lumen of the delivery sheath and aligns below the mitral annulus (2). In some embodiments, the tissue piercing device (10) has a deflectable tip to allow more accurate and easier manipulation and location of the tip of the device relative to the annulus (2). The tip of the tissue piercing device (10) can include a radiopaque marker so that the device may more easily be visualized by using radiographic imaging equipment such as with x-ray, magnetic resonance, ultrasound, or fluoroscopic techniques. FIG. 8a illustrates that the delivery catheter (12) of the tissue piercing device (10) advances distally, and is positioned adjacent, approximate to, or against the mitral annulus at the puncture site. The distal tip (32) of the tissue piercing wire (14) is then pushed against the annulus tissue. In some embodiments, a counter force from the distal tip (32) of the tissue piercing wire (14) is detected. Alternatively, visualization techniques such as, three-dimensional echocardiogram or magnetic resonance imaging, can be used.

In various embodiments of the present teachings, a tissue piercing wire (14) is pre-loaded within the lumen (26) of a delivery catheter (12) during advancement of the delivery catheter (12) to the treatment site. In various other embodiments, the tissue piercing wire (14) is advanced separately after the delivery catheter (12) is placed at the treatment location.

Referring to FIG. 8b, once a delivery catheter (12) is properly positioned, a tissue piercing wire (14) is advanced relative to the delivery catheter (12). According to one embodiment of the present teachings, radio frequency energy is then applied so that the distal tip of the tissue piercing wire (14) is advanced through the annuls (2) and reaches the left atrium. In some embodiments, about 10 mm of the tissue piercing wire (14) extends from the distal end of the delivery catheter (12). Alternatively, about 30 mm of the tissue piercing wire (14) extends from the distal end of the delivery catheter (12). As the distal portion (36) of the tissue piercing wire (14) extends further outside of the delivery catheter (12), the distal portion (36) of the wire transitions to a bended profile to prevent it from causing unnecessary tissue damage inside the left atrium.

In some embodiments, movement of a tissue piercing wire (14) is accomplished manually. Alternatively, movement of a tissue piercing wire (14) may be automated and therefore requires additional controls such as a spring-loaded mechanism, attached to the delivery. Such embodiments are easier for clinician to manipulate and safer for the patient.

The method for tissue piercing described herein is advantageous over the conventional methods. For example, when using the devices and methods of the present teachings, if the distal tip (32) of the wire (14) inadvertently contacts the left atrial free wall (not shown), the floppiness of the distal portion (64) of the conductive inner wire (40), caused by the bendability intermediate portion (62), would not result in damage to or perforation of the left atrial free wall. Another advantage of the transeptal puncture devices (10) described herein is the ability of the device to pierce through thick tissues.

Unless otherwise defined, all technical and scientific terms used herein have the same meaning as commonly understood by one of ordinary skill in the art to which this present teachings belong. Methods and materials similar or equivalent to those described herein can be used in the practice or testing of the present teachings. In case of conflict, the patent specification, including definitions, will control. In addition, the materials, methods, and examples are illustrative only and not intended to be limiting.

We claim:

1. A device for piercing heart tissue percutaneously, comprising:
an outer catheter with a proximal end, a distal end and a lumen longitudinally disposed therethrough;
an inner tissue piercing wire with a conductive inner wire and an insulation outer layer, the inner tissue piercing wire being slidably disposed within the lumen of the outer catheter;
an energy source operatively coupled to the second inner tissue piercing wire; and
wherein the conductive inner wire of the inner tissue piercing wire has a proximal portion, a distal portion, and an intermediate portion, and wherein the intermediate portion is narrower than the proximal portion and distal portion of the conductive inner wire.

2. The device of claim 1, wherein the tissue piercing wire is rotatably movable within the lumen of the outer catheter.

3. The device of claim 1, wherein the insulation outer layer has an open distal end through which the conductive inner wire passes such that that a distal end of the conductive inner wire extends distal to the open distal end of the insulation outer layer.

4. The device of claim 1, wherein the insulation outer layer comprises a coating disposed over the conductive inner wire.

5. The device of claim 1, wherein the insulation outer layer comprises a sleeve.

6. The device of claim 1, further including a coil spring disposed between the conductive inner wire and the insulation outer layer.

7. The device of claim 6, wherein the coil spring covers less than an entire length of the conductive inner wire and is at least disposed along a length of the conductive inner wire that is covered by the insulation outer layer.

8. The device of claim 1, wherein the conductive inner wire is formed of a material selected from the group consisting of; a straight stainless steel wire, a coiled stainless steel wire, a glass fiber, a nitinol material and a polymeric material.

9. The device of claim 1, wherein the energy source is selected from the group consisting of: microwave, infrared, visible light, ultraviolet rays, x-rays, gamma rays, cosmic rays, acoustic energy, thermal energy and radio frequency energy.

10. The device of claim 1, wherein there is a first tapered region between intermediate portion and the proximal portion and there is a second tapered region between the intermediate portion and the distal portion.

11. The device of claim 1, wherein the distal portion has a length between about 5 mm to about 30 mm and the intermediate portion has a length between about 5 mm to about 15 mm.

12. The device of claim 1, wherein the distal portion has a diameter between about 0.1 mm to about 0.3 mm and a diameter of the intermediate portion is between 5-70% of the diameter of the distal portion.

13. The device of claim 1, wherein the distal portion is bent at an angle up to 270 degrees relative to the intermediate portion.

14. The device of claim 13, wherein the bent distal portion is disposed outside of a distal end of the insulation outer layer.

15. A device for piercing heart tissue percutaneously, comprising:
an energy source,
an outer catheter with a proximal end, a distal end and a lumen longitudinally disposed therethrough,
an inner tissue piercing wire with a proximal portion, a distal portion and a intermediate portion between the proximal and distal portions,
wherein the inner tissue piercing wire is configured to transition from a delivery profile, wherein the distal portion of the inner tissue piercing wire is disposed within the lumen of the outer catheter, and substantially aligns with the proximal portion of the inner tissue piercing wire, to a deployed profile, wherein the distal portion of the inner tissue piercing wire is exposed outside of the lumen of the outer catheter, and pivots from the proximal portion of inner tissue piercing wire, and
wherein the energy source is operative coupled to the inner tissue piercing wire.

16. A method for piercing heart valve tissue comprising the steps of:
delivering a delivery catheter to a tissue treatment site by passing the delivery catheter into an aorta through an aortic valve into a left ventricle between cordae tendonae;
delivering a tissue piercing device through a lumen of the delivery catheter to the tissue treatment site, wherein the tissue piercing device comprises:
an outer catheter with a proximal end, a distal end and a lumen longitudinally disposed therethrough;
an inner tissue piercing wire with a conductive inner wire and an insulation outer layer, the inner tissue piercing wire being slidably disposed within the lumen of the outer catheter;
an energy source operatively coupled to the second inner tissue piercing wire; and
wherein the conductive inner wire of the inner tissue piercing wire has a proximal portion, a distal portion, and an intermediate portion, and wherein the intermediate portion is narrower than the proximal portion and distal portion of the conductive inner wire; and
activating the energy source and directing the distal portion against the tissue at the treatment site to cause a piercing thereof.

17. The method of claim 16, wherein the tissue treatment site comprises a mitral annulus.

18. The method of claim 17, wherein the energy source comprises radio frequency energy and a distal tip of the distal portion of the tissue piercing wire is advanced through the annuls and reaches a left atrium.
